(19) United States
(12) Patent Application Publication
(10) Pub. No.: US 2026/0183009 A1
(43) Pub. Date: Jul. 2, 2026

(54) A VISUAL INTERFACE FOR A SYSTEM USED TO DETERMINE TISSUE CHARACTERISTICS

(71) Applicant: Briteseed, LLC, Chicago, IL (US)

(72) Inventors: Jonathan Gunn, Chicago, IL (US); Antonio Belton, Richton Park, IL (US)

(21) Appl. No.: 18/863,848

(22) PCT Filed: May 11, 2023
(86) PCT No.: PCT/US2023/066875

§ 371 Date: Nov. 7, 2024

Related U.S. Application Data
(60) Provisional application No. 63/340,928, filed on May 11, 2022.

(51) Int. Cl. A61B 17/28; A61B 17/00

(52) U.S. Cl. CPC A61B 17/28; A61B 2017/00057; A61B 2017/00115

(57) ABSTRACT

A medical system includes first and second opposing jaws each having an internal surface, at least one light emitter and at least one light sensor each disposed on the internal surface of one of the first and second jaws, and at least one of the first and second jaws having an external surface opposite the internal surface, the external surface having at least one marking disposed thereon aligned with the at least one light sensor. The medical system also includes at least one visual display, and a controller coupled to the light sensor and the visual display. The controller is configured to determine a position of a tissue relative to the light sensor, and control the visual display to display a graphical interface comprising at least one marking corresponding to the marking on the external surface in combination with an image corresponding to the tissue.

### BACKGROUND

This patent is directed to a visual interface for a system used to determine characteristics of tissue, and in particular to a visual interface for a system used to determine characteristics of tissue, where the visual interface includes a surgical device having at least one marking thereon and at least one visual display.

Systems and methods that identify tissues, such as a vessel, in the surgical field during a surgical procedure provide valuable information to the surgeon or surgical team. U.S. hospitals lose billions of dollars annually in unreimbursable costs because of inadvertent vascular damage during surgery. In addition, the involved patients face a mortality rate of up to 32%, and likely will require corrective procedures and remain in the hospital for an additional nine days, resulting in tens, if not hundreds, of thousands of dollars in added costs of care. Consequently, there is this significant value to be obtained from methods and systems that permit accurate determination of the presence of tissues, such as vessels and more particularly blood vessels, in the surgical field, so that these costs may be reduced or avoided.

Further, systems and methods that provide information regarding the presence of tissues in the surgical field are particularly important during minimally invasive surgical procedures. Traditionally, surgeons have relied upon direct visualization and tactile sensation during surgical procedures both to identify tissues, such as blood vessels, and to avoid inadvertent damage to these tissues. Because of the shift towards minimally invasive procedures, including laparoscopic and robotic surgeries, surgeons have lost the ability to use direct visualization and the sense of touch to make determinations as to the tissues present in the surgical field. Consequently, surgeons must make the determination whether tissues are present in the surgical field based primarily on convention and experience. Unfortunately, anatomical irregularities frequently occur because of congenital anomalies, scarring from prior surgeries, and body habitus (e.g., obesity). Systems and methods that would permit surgeons to determine the presence and/or the characteristics of tissues in the surgical field during surgery (potentially in real time or near real time) under such conditions would be a significant advantage.

On the other hand, while it would be advantageous to include systems and methods that provide information regarding the presence of tissues in the surgical field, the adoption of such systems and methods would be impeded if these systems and methods made the surgical procedure more complicated. As mentioned above, the surgeon often would determine the presence and/or characteristics of tissues (e.g., vessels) in the surgical field by direct visualization and/or touch. As such, the surgeon was able to perform several tasks simultaneously by relying on different senses to obtain different information: some information might be obtained visually, other information by touch. By eliminating the surgeon's ability to directly visualize and interact with the surgical field by touch, minimally invasive surgery not only eliminates the ability of the surgeon to use touch to locate, for example, vessels in the surgical field, but to the extent that this information is presented to the surgeon visually, it must compete with all of the other visual tasks that the surgeon must perform for the surgery to be a success. Consequently, if the information were to be provided visually, it would be advantageous if the information were to be provided without the need for an additional video display to be added to the already cluttered bank of equipment that the surgeon or surgical team must monitor during a procedure.

As set forth in more detail below, the present disclosure describes a visual interface embodying advantageous alternatives to the existing systems and methods, which may provide for improved identification for avoidance or isolation of tissues, such as vessels, without undue complication of the surgical instrument or surgical procedure.

### SUMMARY

According to an aspect of the present disclosure, a medical system includes a first jaw having an internal surface and a second, opposing jaw having an internal surface, at least one light emitter disposed on the internal surface of one of the first and second jaws, and at least one light sensor disposed on the internal surface of one of the first and second jaws, and at least one of the first and second jaws having an external surface opposite the internal surface, the external surface having at least one marking disposed on the external surface, and the at least one marking aligned with the at least one light sensor. The medical system also includes at least one visual display, and a controller coupled to the at least one light sensor and the at least one visual display. The controller is configured to determine a position of a tissue relative to the at least one light sensor based on a signal from the at least one light sensor, and control the at least one visual display to display a graphical interface comprising at least one marking corresponding to the at least one marking on the external surface of the at least one of the first and second jaws in combination with an image corresponding to the tissue disposed between the first and second jaws.

### BRIEF DESCRIPTION OF THE DRAWINGS

The disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings is necessarily to scale.

FIG. 1 is a schematic diagram of a surgical system including a surgical instrument, according to an embodiment of the present disclosure;

FIG. 2 is an enlarged, fragmentary view of a transmittance-based embodiment of the surgical instrument of FIG. 1 with light emitters and light sensors, and a section of tissue, including a vessel, illustrated as disposed between the light emitters and light sensors;

FIG. 3 is an enlarged, fragmentary view of a reflectance-based embodiment of the surgical instrument of FIG. 1 with light emitter and light sensor with fixed spacing, and a section of tissue, including a vessel, illustrated as proximate the light emitter and light sensor;

FIG. 4 is an enlarged, fragmentary view of another reflectance-based embodiment of the surgical instrument of FIG. 1 with light emitter and light sensor having an adjustable spacing relative to each other, and a section of tissue, including a vessel, illustrated as proximate the light emitter and light sensor;

FIG. 5 is a schematic diagram of the combination of a surgical instrument, such as may be part of the surgical system of FIG. 1, and a visual display, which surgical instrument and visual display together may define an embodiment of an improved graphical interface;

FIG. 6 is a schematic diagram of the combination of a surgical instrument, such as may be part of the surgical system of FIG. 1, and a visual display, which surgical instrument and visual display together may define another embodiment of an improved graphical interface;

FIG. 7 is a schematic diagram of the combination of a surgical instrument, such as may be part of the surgical system of FIG. 1, and a visual display, which surgical instrument and visual display together may define a further embodiment of an improved graphical interface;

FIG. 8 is a schematic diagram of the combination of a surgical instrument, such as may be part of the surgical system of FIG. 1, and a visual display, which surgical instrument and visual display together may define a still further embodiment of an improved graphical interface;

FIG. 9 is a schematic diagram of the combination of a surgical instrument, such as may be part of the surgical system of FIG. 1, and a visual display, which surgical instrument and visual display together may define yet another embodiment of an improved graphical interface;

FIG. 10 is a schematic diagram of the combination of a surgical instrument, such as may be part of the surgical system of FIG. 1, and a visual display, which surgical instrument and visual display together may define a further embodiment of an improved graphical interface;

FIG. 11 is a schematic diagram of the combination of a surgical instrument, such as may be part of the surgical system of FIG. 1, and a visual display, which surgical instrument and visual display together may define a still further embodiment of an improved graphical interface;

FIG. 12 is a schematic diagram of the combination of a surgical instrument, such as may be part of the surgical system of FIG. 1, and a visual display, which surgical instrument and visual display together may define still another embodiment of an improved graphical interface;

FIG. 13 is a schematic diagram of the combination of a surgical instrument, such as may be part of the surgical system of FIG. 1, and a visual display, which surgical instrument and visual display together may define an additional embodiment of an improved graphical interface;

FIG. 14 is a schematic diagram of the combination of a surgical instrument, such as may be part of the surgical system of FIG. 1, and a visual display, which surgical instrument and visual display together may define a further embodiment of an improved graphical interface;

FIG. 15 is a schematic diagram of a surgical system according to an embodiment of the present disclosure, including an embodiment of a video system;

FIG. 16 is a schematic diagram of a surgical system according to an embodiment of the present disclosure, including another embodiment of a video system; and

FIG. 17 is a partial perspective view of a surgical system according to an embodiment of the present disclosure, incorporating a different surgical instrument than is illustrated in FIG. 1.

### DETAILED DESCRIPTION OF VARIOUS EMBODIMENTS

The embodiments described herein provide medical systems (e.g., surgical systems, according to the illustrated embodiments) with graphical interfaces for use with or in systems used to determine characteristics of tissue(s). These graphical interfaces may include visible markings made on a medical or surgical instrument. A representative of the visible markings may be included in a graphical interface on a visual display to illustrate the position of a tissue (e.g., a vessel) relative to the visible markings on the medical or surgical instrument.

The above surgical system may include a medical instrument (e.g., a surgical instrument, according to the illustrated embodiments) with at least one light emitter and at least one light sensor, and a controller coupled to the at least one light sensor. The controller is configured to determine a position of a tissue relative to the at least one light sensor based on a signal from the at least one light sensor. A variety of different systems including at least one light emitter, at least one light sensor, and an associated controller have been proposed by the applicant for making this determination, using light transmitted through the tissue or reflected from the tissue, as will be explained in greater detail below. One or more of these different systems may be included in the medical instrument.

The medical instrument has a visible surface (i.e., an external surface) with at least one marking disposed on the visible surface. The at least one marking is aligned with the at least one light sensor in a known fashion. The controller is coupled to at least one visual display, and is configured to control the at least one visual display to display a graphical interface comprising at least one marking corresponding to the at least one marking on the external surface of the medical instrument in combination with an image corresponding to the tissue disposed between the first and second jaws.

According to one series of embodiments, the surgical system includes a medical instrument having a first jaw having an internal surface and a second, opposing jaw having an internal surface. As such, the surgical system may be described as having a first jaw having an internal surface and a second, opposing jaw having an internal surface.

The at least one light emitter may be disposed on the internal surface of one of the first and second jaws, and the at least one light sensor may be disposed on the internal surface of one of the first and second jaws. In one series of embodiments, the at least one light emitter may be disposed on the first jaw, and the at least one light sensor may be disposed on the second jaw, the determination of the position of the tissue relying on transmitted light. In another series of embodiments, the at least one light emitter and the at least one light sensor may be disposed on the same jaw (i.e., either the first jaw or the second jaw), the determination of the position of the tissue relying on reflected light.

At least one of the first and second jaws has an external surface opposite the internal surface. The external surface has the at least one marking disposed on the external surface, and the at least one marking is aligned with the at least one light sensor.

By thus combining a marking representative of the marking on the medical instrument with an image corresponding to the tissue, the system displays the information obtained from the sensor such that the information can be correlated with markings present in the surgical field from which the information was obtained. This may simplify the surgeon's or surgical team's processing of this information in one or more of a number of different ways. For example, the information regarding the tissue between the jaw is not displayed in the surgical field, at or near the site of the surgery. This avoids the possibility that fluids (e.g., blood) present in the surgical field might obscure the information regarding the tissue (e.g., its presence and type). In fact, the marking on the tool or instrument may be optimized to facilitate its visibility, even in the presence of bodily fluids and other aspects of the surgery. Likewise, the tissue image may be optimized for its display on the display device. The combination of the marking (more correctly, a marking representative of the marking on the instrument or tool) and the tissue image in the graphical interface provides the possibility that the image of the tissue, which may be optimized for its readability on the visual display, and the marking, which may be optimized for its readability in the surgical field, may provide an overall improved interface for transferring this information to the user (e.g., surgeon). This would be of benefit in minimally invasive and robotic surgery, but could even be of benefit where the surgeon is capable of visualizing the surgical field directly with their eyes.

Having thus discussed the surgical system in general terms, various embodiments of the surgical system are described below. These embodiments are provided for purposes of explanation, and not by way of limitation.

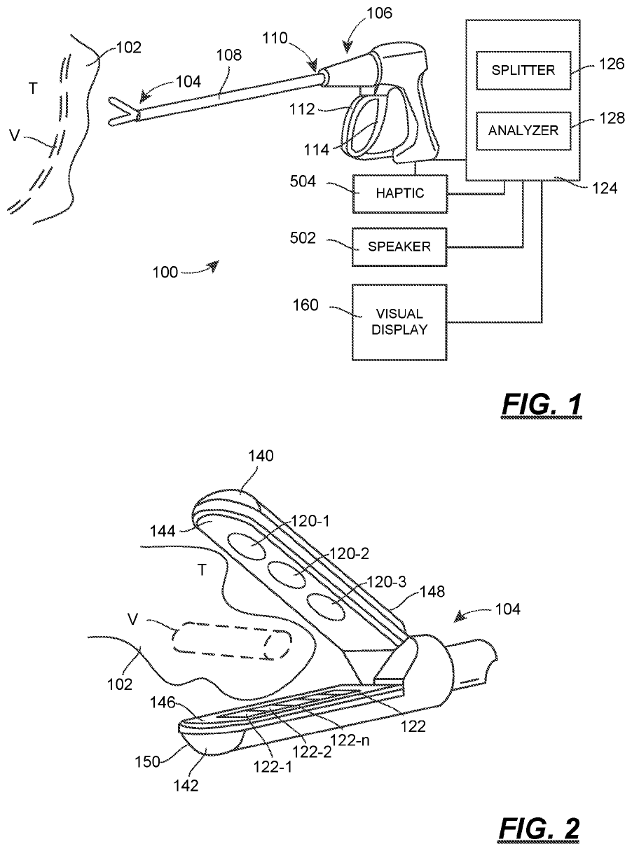

Turning first to FIGS. 1-4, embodiments of a surgical system 100 are illustrated, which system 100 may be used to determine a characteristic (e.g., presence, diameter, etc.) of a tissue. For example, the system 100 may be used to determine the presence of one tissue, such as a vessel, V, within a region 102 of another tissue, T, proximate to a working end 104 of a surgical instrument 106. While the embodiments of FIGS. 1-4 are illustrated with respect to an example where one of the two tissues is vascular tissue, the utility of this system 100 is not limited to such an environment. In addition, the environment is not limited to two tissues, but may include more than two tissues, or may even include a single tissue (e.g., a skeletonized blood vessel).

It will be understood that the vessel V may be connected to other vessels with the region 102 of tissue T, and in addition, the vessel V may extend beyond the region 102 so as to be in fluid communication with other organs also found in the body of the patient (e.g., the heart). Furthermore, while the tissue T appears in FIGS. 1-4 to surround fully the vessel V (in terms of both circumference and length) to a particular depth, this need not be the case in all instances where the system 100 is used. For example, the tissue T may only partially surround the circumference of and/or only surround a section of the length of the vessel V, or the tissue T may overlie the vessel V in a very thin layer. As further non-limiting examples, the vessel V may be a blood vessel, and the tissue T may be connective tissue, adipose tissue and/or liver tissue.

According to the illustrated embodiments in FIGS. 1-4, the working end 104 of the surgical instrument 106 is also a distal end of a shaft 108. Consequently, the working end and the distal end will be referred to as working end 104 or distal end 104. The shaft 108 also has a proximal end 110, and a grip or handle 112 (referred to herein interchangeably as grip 112) is disposed at the proximal end 110 of the shaft 108. The grip 112 is designed in accordance with the nature of the instrument 106; as to the thermal ligation device illustrated in FIG. 1, the grip 112 may be a pistol-type grip including a trigger 114. As a further alternative, finger rings arranged in a generally scissors-type grip may be used.

While the working or distal end 104 and the proximal end 110 with grip 112 are illustrated as disposed at opposite-most ends of the shaft 108, it will be recognized that certain surgical instruments have working ends (where a tool tip is attached, for example) disposed on the opposite-most ends of the shaft and a gripping region disposed intermediate to the opposite working ends. In accordance with the terms “distal” and “proximal” as used herein, the working ends of such an instrument are referred to herein as the distal ends and the gripping region as the proximal end. Relative to the illustrated embodiments, however, the distal and proximal ends are located at opposite-most (or simply opposite) ends of the shaft 108.

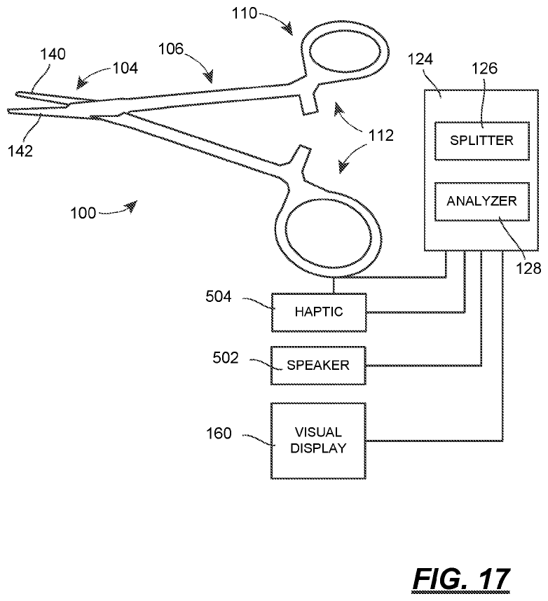

It will also be recognized that while the surgical instrument 106 is illustrated as including a shaft 108, the embodiments of the system 100 are not limited to only instruments 106 that have an elongated shaft such as is illustrated. For example, the instrument 106 may be in the form that resembles a scissors-type tool (e.g., forceps, hemostat, sealer/divider, etc.), in which case one may still refer to a distal or working end 104 and a proximal end 110 where a grip 112 (in the form of finger rings or handles) may be disposed. An illustration of such an embodiment is included at FIG. 17, numbered consistently with the embodiment illustrated in FIG. 1. Other embodiments, including embodiments where the working end 104 is part of a robotic instrument, are also within the scope of the present disclosure.

As mentioned above, according to the illustrated embodiments, the surgical system 100 includes a sensor with at least one light emitter 120 (or simply the light emitter 120) and at least one light sensor or detector 122 (or simply the light sensor 122). See FIGS. 2-4. According to the illustrated embodiments, a controller 124 is coupled to the light emitter 120 and the light sensor 122, which controller 124 may include a splitter 126 and an analyzer 128 as explained below. See FIGS. 1 and 17.

The light emitter 120 is disposed at the working end 104 of the surgical instrument 106. The light sensor 122 is also disposed at the working end 104 of the surgical instrument 106. Either the light emitter 120 or the light sensor 122 may be referred to as disposed at the working end 104 where the light emitter 120 or the light sensor 122 is physically mounted at the working end 104. Alternatively, the light emitter 120 or the light sensor 122 may be referred to as disposed at the working end 104 where the light emitter 120 or light sensor is connected by a light guide (e.g., fiber optics), with a first end of the light guide disposed at the working end 104 and a second end of the light guide disposed elsewhere (e.g., at the proximal end 110).

The system 100 may operate according to a transmittance-based approach, such that the light sensor(s) 122 is/are disposed opposite and facing the light emitter(s) 120, for example on opposite jaws 140, 142 of a surgical instrument 106 as illustrated in FIG. 2 (or in FIG. 17). More particularly, the first jaw 140 may have an internal surface 144 on which the at least one light emitter 120 is disposed or mounted, and the second, opposing jaw 142 may have an internal surface 146 on which the at least one light sensor 122 is disposed or mounted. It is also possible that the system 100 may operate according to a reflectance-based approach, such that the light sensor(s) 122 is/are disposed on the same structure (e.g., jaw) as the light emitter(s) 120 with the light sensor and emitter 122, 120 facing in a common direction, with the resultant structure appearing quite similar to the user as the transmittance based approach illustrated in FIG. 2. For example, both the light emitter 120 and light sensor 122 may be disposed on the internal surface 146 of the second jaw 142.

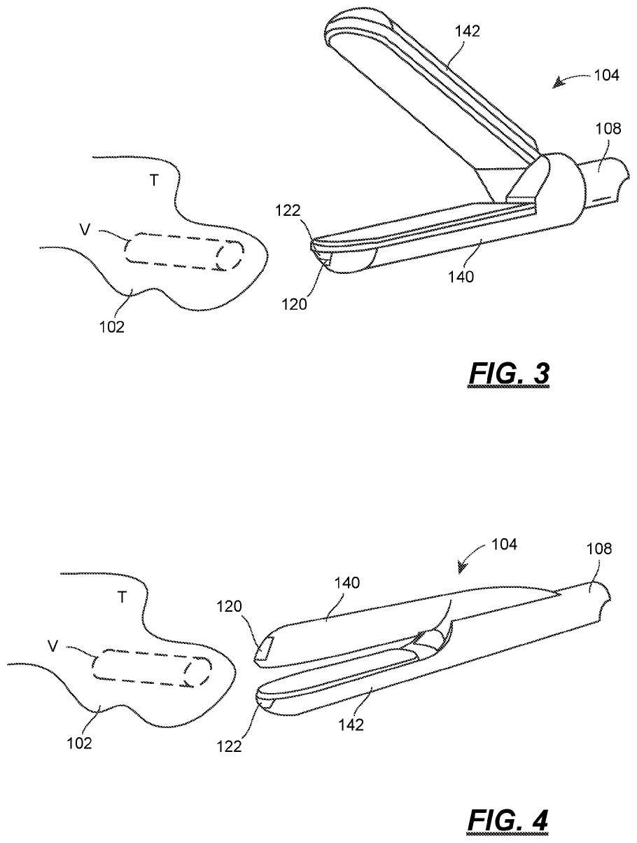

It is also possible to have the system 100 operate according to a reflectance-based approach, such that the light emitter 120 and the light sensor 122 may face in a common direction and with fixed spacing therebetween, for example on a single jaw 140 of a two-jaw device 140, 142, such as a thermal ligation device (FIG. 3), although the relative angle between the light emitter 120 and light sensor 122 may be fixed or variable. The light emitter 120 and the light sensor 122 of a reflectance-based system may be constructed such that the spacing between the light emitter 120 and the light sensor 122 may be adjusted, for example by positioning the light emitter 120 at the end or tip of one of the jaws 140 of a two-jaw device and the light sensor 122 at the end or tip of the other the jaws 142 of the two-jaw device, as illustrated in FIG. 4.

The light emitter 120 may be adapted to emit light of at least one wavelength. For example, the light emitter 120 may emit light having a wavelength of 660 nm. This may be achieved with a single element, or a plurality of elements (which elements may be arranged or configured into an array, for example, as explained in detail below). In a similar fashion, the light sensor 122 is adapted to detect light at the at least one wavelength (e.g., 660 nm). According to the embodiments described herein, the light sensor 122 also may include a plurality of elements, which elements are arranged or configured into an array.

According to certain embodiments, the light emitter 120 may be configured to emit light of at least two different wavelengths, and the light sensor 122 may be configured to detect light at the at least two different wavelengths. As one example, the light emitter 120 may emit and the light sensor 122 may detect light of multiple wavelengths in the visible range and light of multiple wavelengths in the near-infrared or infrared range. According to other embodiments, the emitter 120 and sensor 122 may emit and detect light of a plurality of wavelengths.

According to some embodiments, the individual light sensor 122 is adapted to generate a signal comprising a first pulsatile component and a second non-pulsatile component. It will be recognized that the first pulsatile component may be an alternating current (AC) component of the signal, while the second non-pulsatile component may be a direct current (DC) component. Where the light sensor 122 is in the form of an array, the pulsatile and non-pulsatile information may be generated for each element of the array, or at least for each element of the array that defines the at least one row of the array.

According to such embodiments, the controller 124 is coupled to the light sensor 122, and may include a splitter 126 to separate the first pulsatile component from the second non-pulsatile component for each element of the light sensor array 122. The controller 124 may also include an analyzer 128 to determine the presence of and/or characteristic(s) of tissue, such as the vessel V, within the region 102 proximate to the working end 104 of the surgical instrument 106 based (at least in part) on the pulsatile and/or the non-pulsatile component. The pulsatile component, the non-pulsatile component, or a combination of both components may be used to determine the characteristics (e.g., presence, measurements) of tissue in the surgical field. Such systems are described in one or more of the following applications, all of which are incorporated by reference herein in their entirety: U.S. Pub. Nos. 2021/0338260; 2021/0068856; 2020/0345297; 2020/0337633; 2020/0268311; 2019/0175158; 2019/0046220; 2019/0038136; 2018/0289315; 2018/0098705; 2018/0042522; 2017/0367772; 2017/0181701; and 2015/0066000.

While the illustrated embodiments utilize a sensor that includes a light emitter(s) and a light sensor(s), the surgical system with graphical interface may be used with other sensors or sensor systems/assemblies. For example, the sensor may include other optical sensors or sensing systems, ultrasound sensors or sensing systems, ultrasound Doppler sensors or sensing systems, acoustic Doppler sensors or sensing systems, laser Doppler sensors or sensing systems, photoacoustic sensors or sensing systems, magnetic sensors or sensing systems, thermographic sensors or sensing systems, sonographic sensors or sensing systems, electrical (e.g., impedance-based) sensors or sensing systems, or any other sensors or sensing systems that may be used to detect or determine characteristics of tissue. Where the sensor or sensing system includes a transmitter (like the light emitter) and a receiver (like the light sensor), the transmitter and receiver may be disposed at the working end 104 of the medical (e.g., surgical) instrument or tool 106 (as that phrased is used herein) like the embodiments illustrated in FIGS. 2-4, above. The graphical interface described herein may be particularly relevant to the light emitter/light sensor based system illustrated herein, but it may be useful with the other sensors or sensing systems described in this paragraph as well.

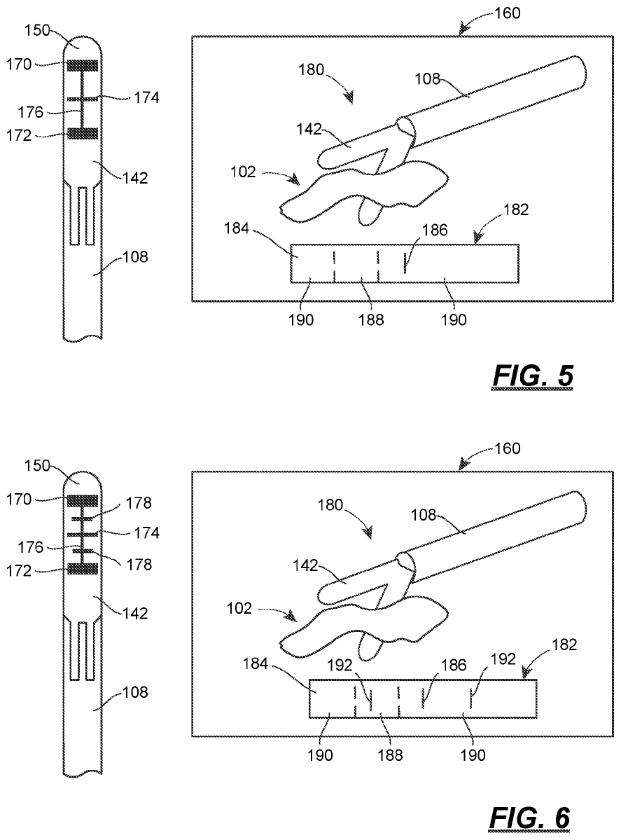

As mentioned above, the medical system according to the present disclosure combines a marking representative of at least one marking on a medical instrument with an image of a tissue determined by the controller to provide a graphical interface for transferring or conveying information to a user. FIGS. 5-14 illustrate different embodiments of such a system. It will be recognized that while each of the different embodiments of FIGS. 5-14 may be considered as the combination of features illustrated only in that specific embodiment, the embodiments of different figures also may share common or overlapping features as well. For example, more than one of the embodiments illustrated in FIGS. 5-14 may include a first marking corresponding to a first end of a light sensor array, a second marking corresponding to a second end of the light sensor array, and a third marking corresponding to a center of the array. As such, it will be recognized that the features of the individual embodiments may also be combined in additional ways not directly illustrated in FIGS. 5-14, which additional ways are consistent with the various embodiments illustrated and the features common or overlapping between the various embodiments.

In a similar way, the description of an embodiment with respect to one surgical instrument or tool is not meant to imply only use with such a surgical instrument or tool. For example, one will recognize while embodiments of FIGS. 5-16 illustrate the graphical interface with a two-jawed instrument or tool where the jaws are disposed at the end of a shaft (as may be the case with an endoscopic or robotic tool), but the two-jawed instrument or tool could be a forceps, hemostat, or sealer/divider instead. As such, any of the embodiments illustrated with reference to the system as illustrated in FIG. 1 incorporating a two-jawed tool having an elongated shaft could also be used in a system as illustrated in FIG. 17.

Each of the embodiments illustrated in FIGS. 5-14 includes an illustration of a plan view of an external surface 148, 150 of at least one of the jaws 140, 142 of a two jaw surgical instrument, in particular a surgical instrument, such as a thermal ligation device. Compare FIG. 2 and FIGS. 5-14. Further, the jaw (e.g., jaw 142) having the marking(s) disposed thereon (e.g., on the external surface 150) also may have a light sensor 122 in the form of a light sensor array disposed on the internal surface (e.g., internal surface 146) opposite the external surface illustrated. According to certain embodiments, the marking(s) may cover an area on the external surface (e.g., 150) that is equal or approximately equal to an area on the internal surface (e.g., 146) covered by the light sensor 122.

According to other embodiments, the marking(s) may cover an area on the external surface that is larger than or smaller than an area on the internal surface that is covered by the light sensor 122.

According to certain embodiments, the marking(s) may be etched on the surface. According to other embodiments, the marking(s) may be disposed on the surface by overlaying (e.g., painting) the marking(s) on the surface instead. Both etching and overlaying may be combined in a single embodiment to dispose the marking(s) on the surface. One method may be more suitable over another depending on the material used for the jaw(s); for example, where the jaws are made of metal, it may be more suitable to etch the markings on the jaws.

According to certain embodiments, it may also be possible to illuminate the marking(s), in addition to or in substitution for etching or overlaying the markings According to certain embodiments, the marking(s) may be defined by a portion (or portions) or region (or regions) of the external surface (e.g., 150) that is transparent or translucent, and one or more light sources (e.g., light emitting diodes (LEDs), an end of a optic fiber, etc.) may be disposed behind the portions of the external surface that is transparent or translucent. In such embodiments, the transparent or translucent portion or region may be defined by removing the material of the jaw (e.g., jaw 142) and replacing the removed material with the transparent or translucent material (like a window). Alternatively, the jaw (e.g., 142) may be made of transparent or translucent material, and the portion or region that will define the marking(s) will be set off by covering the remainder of the external surface with a substance that does not allow light to pass through (i.e., that is opaque, or at least less translucent than the area forming the marking(s)), such as a mask, shield, or coating. According to other embodiments, the marking(s) may be defined by the light sources themselves: for example, LEDs may be disposed on an external surface or may be mounted in the external surface to define the marking(s).

Each of FIGS. 5-14 also illustrates a visual display 160 that is used in conjunction with the surgical instrument illustrated. While the visual display could be part of a video monitor, for example, it is also possible that the visual display could be part of a heads up video display, video headset, or pair of smart glasses, for example. Moreover, the present disclosure is not limited to an embodiment where a single visual display 160 is used. Multiple visual displays may be used, and certain of the displays may display only a graphical interface as explained in greater detail below, while other of the displays display the graphical interface with additional information. As another example, the graphical interface may be displayed as a picture-in-picture along with other information on the patient's vital signs, or vice versa.

Starting then with FIG. 5, an embodiment of the surgical system 100 is illustrated with a surgical instrument 106, such as may be explained relative to the embodiment of FIG. 2, for example. In the embodiment of FIG. 2, the surgical instrument 106 has two jaws 140, 142, with the at least one light emitter 120 disposed on (or more particularly mounted on) the jaw 140 and with the at least one light sensor 122 disposed on (or more particularly mounted on) the jaw 142. As such, the instrument 106 is illustrated in the left half of FIG. 5 with the external surface 150 in plan view, having markings 170, 172, 174, 176 disposed on the external surface 150. According to other embodiments, both jaws 140, 142 may have a marking or marking(s), such as the markings 170, 172, 174, 176 disposed on their respective external surfaces 148, 150 instead.

The markings 170, 172, 174, 176 are arranged on or formed on the external surface 150 such that the transverse marking 174 is aligned with the at least one light sensor 122. In particular, where the at least one light sensor 122 is an array of light sensors 122, the marking 174 disposed on the external surface 150 corresponds to the center of the array. In fact, where the at least one light sensor 122 is a linear array of light sensors 122, the marking 174 is disposed on the external surface 150 at the middle of the linear array. This marking 174 may also be referred to as the transverse central axis.

In addition, the transverse marking 170 is disposed on the external surface 150 at a first end of the array of light sensors 122, while the transverse marking 172 is disposed on the external surface 150 at a second end of the array of light sensors 122. The transverse marking 174 is thus equally distant from the markings 170, 172 because the marking 174 corresponds to the center or middle of the array of light sensors 122. All three of the transverse markings 170, 172, 174 may be lines of different thicknesses, which lines may appear almost rectangular in shape relative to the other lines because of the relative thicknesses, and may be referred to interchangeably as bars. The differing thicknesses may be used to differentiate between the markings in the interior (between the ends of the set of markings) and the markings in the exterior (at the ends of the set of markings).

As also illustrated in the embodiment of FIG. 5, the markings may include a marking 176 that connects the three markings 170, 172, 174 mentioned above. The marking 176 may represent a longitudinal axis of the array of light sensors 122, and may be disposed along or through a midpoints of each of the markings, or lines, 170, 172, 174 as a longitudinal central axis. According to other embodiments, the marking 176 may be disposed either at one end or the other of the lines 170, 172, 174, or may be disposed closer to one of the ends of the lines 170, 172, 174 than the other (i.e., more to the left or right than is illustrated in FIG. 5, with reference to the orientation of the jaw 142 in the plan view of FIG. 5). Because the line 176 of the embodiment of FIG. 5 is disposed interior of the end lines 170, 172, it is also thinner than the end lines 170, 172, like the center line 174.

The visual display 160 is illustrated in the right half of FIG. 5, and may include a live image 180 of the surgical field 102 and a graphical interface 182. The controller 124 may combine the live image 180, as received from a camera or scope, with the graphical interface 182 to provide an integrated image that includes both a visual image of the surgical field and information derived from the light sensor(s) 122. In particular, the controller 124 may determine a position of a tissue relative to the at least one light sensor 122 based on a signal from the at least one light sensor 122, and then control the visual display to display the graphical interface 182 that provides an image 184 corresponding to the calculated position of the tissue between the jaws 140, 142. More particularly, the controller 124 may control the visual display 160 to display a graphical interface including at least one marking 186 corresponding to the at least one marking 174 on the external surface 150 of the second jaw 142 in combination with the image 184 corresponding to the tissue disposed between the first and second jaws 140, 142.

In the embodiment illustrated in FIG. 5, the image 184 includes at least two different regions 188, 190. The region 188 includes the area between the dashed lines, and this region may represent a first tissue type, e.g., a blood vessel. The region 190 includes the areas outside the dashed lines, and this region may represent a second tissue type, e.g., adipose tissue. The two regions may be differentiated in the image 184 through the use of different colors; for example, the region 188 may be filled in red, while the region 190 may be filled in white. Other schemes may be used to differentiate the different regions apart (e.g., different shades of a single color), and the regions 188, 190 may include a single region (e.g., corresponding to only adipose tissue disposed between the jaws 140, 142 of the instrument 106) or more than two regions (e.g., a ureter, a blood vessel, and adipose tissue).

As also seen in the graphical interface 182, the marking 186 may include a line or a bar, although other geometric shapes may be used instead. The marking 186 corresponds to the center line 174 on the markings on the instrument 106, and in particular the jaw 142. When combined with the image 184, the marking 186 may convey to the user the information that the vessel (as represented by region 188) is between the marking 174 and the marking 170, the marking 170 having been previously indicated to the user to correspond to the left end of the graphical interface 182. To remind the user of this correspondence, the interface 182 may have a left end that his more rounded in shape than the flat end illustrated in FIG. 5. See FIG. 8, described below.

Other information may be combined with the graphical interface 182. For example, the width of the vessel may be displayed at one end or the other of the graphical interface 182. Alternatively, a numerical scale may be displayed along the image 184 to permit the user to determine the relative distances between the tissues and the center marking 174 or the end markings 170, 172. See, e.g., FIG. 8.

FIG. 6 illustrates an embodiment similar to that illustrated in FIG. 5. As such, the numbering for common features will be retained from FIG. 5, while the new numbering will be used for features unique to the embodiment of FIG. 6.

The embodiment of FIG. 6 includes additional markings 178 disposed between the first and third markings 170, 174 and the second and third markings 172, 174, the additional markings 178 representing different dimensions than the first, second, and third markings 170, 172, 174. For example, the markings 178 may be disposed equally distant from the first and third markings 170, 174 and the second and third markings 172, 174, and may represent half the distance between the major markings 170, 172, 174. These additional markings 178 may be referred to as quadrant demarcations. The markings 178 may be thinner than the markings 170, 172 because they are interior to the ends of the set of markings, and may be shorter transversely than the transverse center axis 174 to differentiate themselves more easily from the center axis 174.

In a similar fashion, the graphical interface 182 includes markings 192 that correspond to the additional markings 178 on the jaw 142, and in particular the external surface 150 of the jaw 142. These additional markings 192 permit the user to further associate the information displayed as the image 184 to the tissue disposed between the jaws 140, 142. The other information discussed above relative to the graphical interface 182 applies equally with respect to the embodiment of FIG. 6.

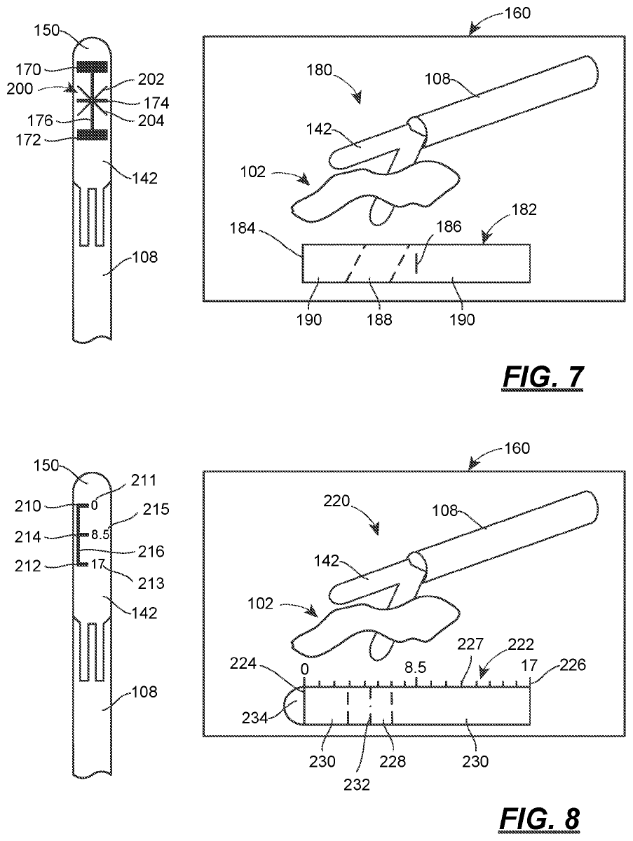

FIG. 7 also illustrates an embodiment similar to that illustrated in FIG. 5. As such, the numbering for common features will be retained from FIG. 5, while the new numbering will be used for features unique to the embodiment of FIG. 7.

The embodiment of FIG. 7 includes a design 200 superimposed on the third marking 174. The design 200 may include a first diagonal 202 and a second diagonal 204 joined at the midpoints to form an “X” that is superimposed on the transverse center axis. The first and second markings 170, 172 still define the extents of the sensor area (or array, according to the illustrated embodiments), and the longitudinal center axis 176 still references with sensor array central plane. However, the diagonals 202, 204 may provide an angular reference to be used in correlating the information of the image 184 to the jaws 140, 142.

To this end, the graphical interface 182 may include data relative to the orientation of the vessel relative to a transverse axis. For example, the region 188 may be disposed at an angle of approximately 20 degrees to a transverse axis. The diagonals 202, 204 may be used by the user as a further reference for the information displayed in image 184 relative to the jaws 140, 142 of the instrument 106. That is, an angle value of 20 degrees may mean that the vessel lies between the jaws 140, 142 in the same orientation as the diagonal 204, but at a shallower slope relative to the transverse axis. The other information discussed above relative to the graphical interface applies equally with respect to the embodiment of FIG. 7.

It will be recognized that the features of the embodiment of FIG. 7 may be combined with the features of the embodiment of FIG. 6. According to such an embodiment, the user would be able to obtain additional spatial information regarding the position of the tissue(s) relative to the center and ends of the sensor array, as well as relative angular information regarding the tissue(s).

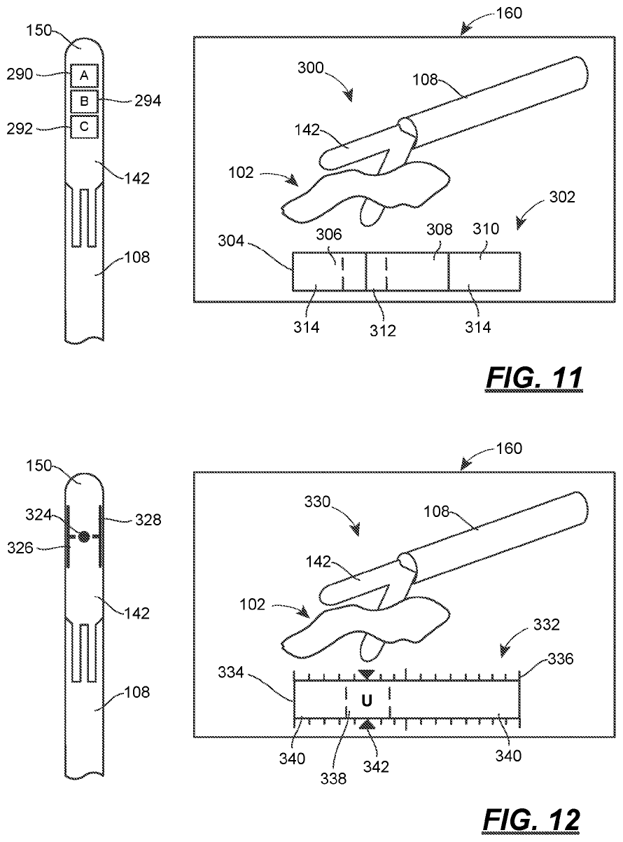

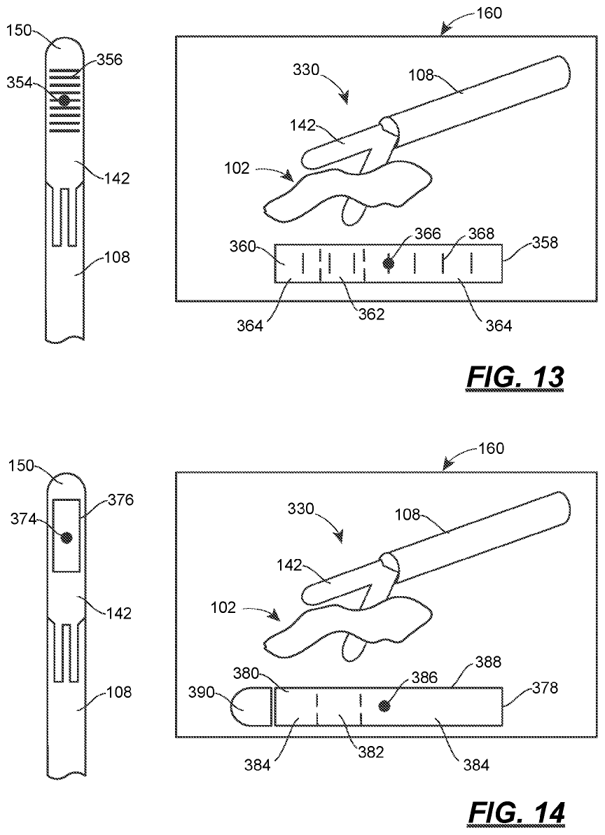

It also will be recognized that the superimposed design may not be used to convey additional information, such as angular information. Instead, the superimposed design may simply used to further highlight the special nature of the transverse central axis 174. See, e.g., FIGS. 12 and 13.

FIG. 8 illustrates an additional embodiment that may be explained relative to the embodiment of the surgical instrument 106 of FIG. 2, for example. As such, the instrument 106 is illustrated in the left half of FIG. 8 with the external surface 150 in plan view, having markings disposed on the external surface 150. According to certain embodiments, both jaws 140, 142 may have a marking, such as the markings 210, 212, 214, 216 disposed on their respective external surfaces 148, 150.

The markings 210, 212, 214, 216 are arranged on or formed on the external surface 150 such that the transverse marking 214 is aligned with the at least one light sensor 122. In particular, where the at least one light sensor 122 is an array of light sensors 122, the marking 214 corresponds to the center of the array, and where the at least one light sensor 122 is a linear array, the marking 214 is disposed at the middle of the linear array. The transverse marking 210 is disposed at a first end and the transverse marking 212 is disposed at a second end of the array of light sensors 122. All three of the transverse markings 210, 212, 214 may be lines of similar thicknesses, because of the related information displayed with each of the lines 210, 212, 214.

As also illustrated in the embodiment of FIG. 8, a marking 216 connects the three markings 210, 212, 214 mentioned above. The marking 216 may represent a longitudinal axis of the array of light sensors 122, and may be disposed at one end or to one side of the first, second, and third markings 210, 212, 214. Like the lines 210, 212, 214, this line 216 may be of a common line weight as the other lines 210, 212, 214.

Each of the markings 210, 212, 214 may be associated or paired with a numerical value 211, 213, 215. This numerical value 211, 213, 215 may correspond to a distance between the marking 210, 212, 214 and one end of the sensor array or the other, as is the case in the illustrated embodiment of FIG. 8. That is, the marking 210 corresponds to the first end of the array, and this is associated with a “0” (zero) value. The second marking 212 is associated with an “17.0” value, corresponding to 17 mm from the end of the array aligned with the marking 210. Similarly, the third marking 214 is associated with a “8.5” value, corresponding to 8.5 mm from the end of the array aligned with the marking 210. The numerical markings 211, 213, 215 may aid in associating the information from the display 160 with the markings 210, 212, 214 on the instrument 106.

It will be recognized that having the numerical markings 211, 213, 215 start at a “0” (zero) value corresponding with marking 210 and increasing in value for each of markings 212, 214 is but one possible option. According to other embodiments, the numerical markings 211, 213, 215 may instead start at marking 212 and increase in value for each of markings 214, 210, respectively. As a further alternative, the numerical markings 211, 213, 215 may be with reference to the marking 214, and indicate the distance from the marking 214. See FIG. 10.

The visual display 160 is illustrated in the right half of FIG. 8, and may include a live image 220 of the surgical field 102 and a graphical interface 222. The controller 124 may combine the live image 220, as received from a camera or scope, with the graphical interface 222 provide an integrated image that includes both a visual image of the surgical field with information derived from the light sensor(s) 122. Similar to the embodiments above, the controller 124 may determine a position of a tissue, and then control the visual display to display the graphical interface 222 that provides an image 224 corresponding to the position of the tissue between the jaws 140, 142. More particularly, the controller 124 may control the visual display 160 to display a graphical interface 222 including at least one marking 226.

In the embodiment illustrated in FIG. 8, the image 224 includes at least two different regions 228, 230. The region 228 includes the area between the dashed lines, and this region may represent a first tissue type, e.g., a blood vessel. The region 230 includes the areas outside the dashed lines, and this region may represent a second tissue type, e.g., adipose tissue. The two regions may be differentiated in the image 224 through the use of different colors, for example, the region 228 may be filled in red, while the region 230 may be filled in green.

According to this embodiment, the marking 226 includes a scale disposed to one side of the image 224. The scale 226 includes a plurality of individual markings 227, each marking 227 corresponding an individual unit of distance from the preceding (or succeeding) marking. To permit the scale to be compared to the markings on the instrument 106, each of the numerical markings 211, 213, 215 may be included in the scale 226. When combined with the image 224, the scale 226 and its graduated length markings may be used to approximate the width of the regions 228, 230 of the image 224.

In addition, the graphical interface 222 may include additional information, such as the width of the tissue within region 228, for example. The interface 222 may also include information on the relative inclination of the tissue within, for example, region 228 relative to a transverse axis. This information may be conveyed both in the form of a numeric value, and in the form of an angle indicator (e.g., a line) 232 superimposed on the region 228.

As a further feature, the graphical interface 222 may include a jaw indicator 234. In the particular embodiment illustrated, the jaw indicator 234 may be a semicircular region that is attached to one end or the other of the tissue image 224. The indicator 234 corresponds to the curved end of the jaw 142, and provides a visual reference for the graphical interface 222 to remind the user of the orientation of the image 224 and the scale 226 to the jaw 142 of the instrument 106.

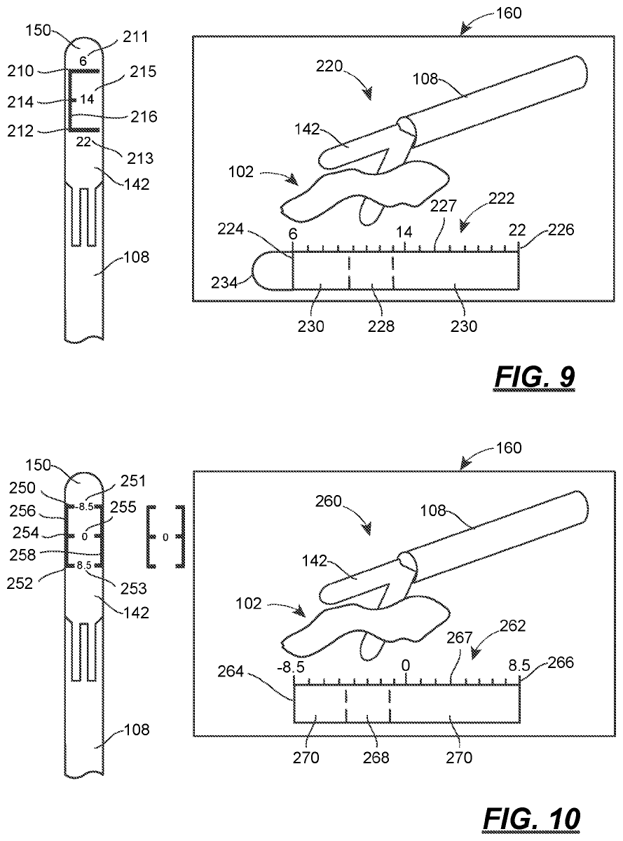

FIG. 9 illustrates an embodiment similar to that illustrated in FIG. 8. As such, the numbering for common features will be retained from FIG. 8, while the new numbering will be used for features unique to the embodiment of FIG. 9.

The markings 210, 212, 214, 216 are arranged on or formed on the external surface 150 such that the transverse marking 214 is aligned with the at least one light sensor 122. In particular, where the at least one light sensor 122 is an array of light sensors 122, the marking 214 corresponds to the center of the array, and where the at least one light sensor 122 is a linear array, the marking 214 is disposed at the middle of the linear array. The transverse marking 210 is disposed at a first end and the transverse marking 212 is disposed at a second end of the array of light sensors 122. All three of the transverse markings 210, 212, 214 may be lines of similar thicknesses, because of the related information displayed with each of the lines 210, 212, 214, but the markings 210, 212 are longer in the transverse direction than the marking 214 so as to indicate the ends of the corresponding sensor array.

As also illustrated in the embodiment of FIG. 8, a marking 216 connects the three markings 210, 212, 214 mentioned above. The marking 216 represents a longitudinal axis of the array of light sensors 122, and is disposed at one end or to one side of the first, second, and third markings 210, 212, 214.

Each of the markings 210, 212, 214 may be associated or paired with a numerical value 211, 213, 215. Unlike the embodiment of FIG. 8, each marking corresponds to a distance between the marking 210, 212, 214 and an end or tip of the jaw 142. That is, the marking 210 corresponds to the first end of the array, and this is associated with a “6” value, representing the fact that the first end of the array (and thus the marking 210) is 6 mm from the end or tip of the jaw 142. The second marking 212 is associated with an “22” value, corresponding to 22 mm from the end of the jaw 142. Similarly, the third marking 214 is associated with a “14” value, corresponding to 14 mm from the end of the jaw 142.

The graphical interface 222 thus conveys information regarding the position of the tissues relative to the end or tip of the jaw, rather than the end of the light sensor array 122. Other than this difference, the general structure and operation of the graphical interface is the same as in FIG. 8.

It will be recognized that features of the embodiments of FIGS. 8 and 9 may be substituted or combined. For example, longer transverse lines from the embodiment of FIG. 9 may be used with the embodiment of FIG. 8 to represent the ends of the light sensor array, but in combination with the numerical values of the embodiment of FIG. 8 illustrating the distances from one end of the light sensor array. As another example, the numerical values from the embodiment of FIG. 9 may be used with the other markings of the embodiment of FIG. 8 to convey the distances from the end or tip of the jaw 142, instead of the distances from an end of the light sensor array.

FIG. 10 is further embodiment with aspects in common with the embodiments of FIGS. 8 and 9, and with new features that provide a substantially different representation overall. That is, the markings of the embodiment of FIG. 10 similarly include a number of transverse markings, as well as at least one longitudinal marking that connects the transverse markings at a first end or to a first side. A second longitudinal marking also connects the transverse markings at a second or opposite end or a second or opposite side as well. As such, the markings form a graphic box that demarks the ends and sides of the light sensor array relative to the external surface 150 of the jaw 142.

Furthermore, the numerical value markings associated with the transverse markings are provided in two variants, as illustrated in the left half of FIG. 10. The first variant, which is illustrated disposed on the surface 150, includes numerical markings that indicate the distance of either end from the transverse center axis, which is associated with a numerical value of “0” (zero). The second variant, which is illustrated just to the right of the variant disposed on the surface 150, does not include numerical markings that indicate the distance of either end from the transverse center axis, but the transverse center axis is marked with a numerical value of “0” (zero).

In a similar fashion, the visual display has a graphical interface that is marked with a scale with graduated distance markings with a central reference location associated with a numerical marking of “0” (zero). As such, the correspondence of between the markings on the external surface 150 of the jaw 142 and those of the graphical interface may be conveyed to the user. Further, the ends of the scale of the graphical interface may include numerical values corresponding to those disposed on the external surface 150, or may be omitted where the numerical values have been omitted on the external surface 150 of the jaw 142.

Starting then at the left hand side of FIG. 10, markings 250, 252, 254, 256, 258 are arranged on or formed on the external surface 150 such that at least the transverse marking 254 is aligned with the at least one light sensor 122. In particular, where the at least one light sensor 122 is an array of light sensors 122, the marking 254 corresponds to the center of the array, and where the at least one light sensor 122 is a linear array, the marking 254 is disposed at the middle of the linear array. The first transverse marking 250 is disposed at a first end and the transverse marking 252 is disposed at a second end of the array of light sensors 122. All three of the transverse markings 250, 252, 254 may be lines of similar thicknesses, because of the related information displayed with each of the lines 250, 252, 254.

As also illustrated in the embodiment of FIG. 10, a first longitudinal marking 256 connects the three markings 250, 252, 254 mentioned above. The marking 256 may be disposed at one end or to one side of the first, second, and third markings 250, 252, 254. A second longitudinal marking 258 also connects the three markings 250, 252, 254 mentioned above. The marking 258 may be disposed at an end or to a side of the first, second, and third markings 250, 252, 254 opposite the first end or side. Like the lines 250, 252, 254, the lines 256, 258 may be of a common line weight as the other lines 250, 252, 254.

As mentioned above, the lines 250, 252, 256, 258 may define a box that demarks the outer boundaries of the light sensor array relative to the external surface 150 of the jaw. It will be recognized that the nature of the external surface 150 of the jaw 142 may make the correspondence only an approximate, in that the internal surface 146 of the jaw 142 may be planar, while the external surface 150 of the jaw 142 may be curved. However, useful information may still be conveyed to the user as a consequence.

Each of the markings 250, 252, 254 of the first variant of the embodiment of FIG. 10 may be associated or paired with a numerical value 251, 253, 255. This numerical value 251, 253, 255 may be correspond to a distance between the marking 250, 252, 254 and the center of the light sensor array. That is, the third marking 254 corresponds to the center of the array, and this is associated with a “0” (zero) value. The first marking 250 is associated with an “−8.5” value, corresponding to 8.5 mm from the center of the array in the direction of an end or tip of the jaw 142. Similarly, the second marking 254 is associated with a “8.5” value, corresponding to 8.5 mm from the center of the array in the direction of a pivot between the jaws 140, 142. Thus, the numerical markings 251, 253, 255 aid in associating the information from the display 160 with the markings 250, 252, 254 on the instrument 106 in that one marking 251 is disposed closer to the end or tip of one of the first and second jaws 140, 142 and another marking 253 is disposed closer to the pivot between the first and second jaws 140, 142, and the marking 251 is different from the marking 253.

It will be recognized that the embodiment of the first variant with numerical markings 251, 253, 255 is but one possible option. According to the embodiment of the second variant, the numerical markings 251, 253 may be omitted. As such, the box defined by the markings 250, 252, 256, 258 remains, but only the transverse center axis is indicated by the numerical marking 255. See, e.g., the variant illustrated in FIG. 10.

The visual display 160 is illustrated in the right half of FIG. 10, and may include a live image 260 of the surgical field 102 and a graphical interface 262. The controller 124 may combine the live image 260, as received from a camera or scope, with the graphical interface 262 provide an integrated image that includes both a visual image of the surgical field with information derived from the light sensor(s) 122. Similar to the embodiments above, the controller 124 may determine a position of a tissue, and then control the visual display to display the graphical interface 262 that provides an image 264 corresponding to the position of the tissue between the jaws 140, 142. More particularly, the controller 124 may control the visual display 160 to display a graphical interface 262 including at least one marking 266.

In the embodiment illustrated in FIG. 10, the image 264 includes at least two different regions 268, 270. The region 268 includes the area between the dashed lines, and this region may represent a first tissue type, e.g., a blood vessel. The region 270 includes the areas outside the dashed lines, and this region may represent a second tissue type, e.g., adipose tissue. The two regions may be differentiated in the image 264 through the use of different colors; for example, the region 268 may be filled in red, while the region 270 may be filled in white.

According to this embodiment, the marking 266 includes a scale disposed to one side of the image 264. The scale 266 includes a plurality of individual markings 267, each marking 267 corresponding an individual unit of distance from the preceding (or succeeding) marking. To permit the scale to be compared with the markings on the instrument 106, at least the numerical marking 255 (“0”) may be included in the scale 266. When combined with the image 254, the scale 266 and its graduated length markings may be used to approximate the width of the regions 268, 270 of the image 264.

In addition, the graphical interface 262 may include additional information, such as the width of the tissue within region 268, for example. The interface 262 may also include information on the relative inclination of the tissue within, for example, region 268 relative to a transverse axis.

The embodiments of FIGS. 11-14 differ from the forgoing embodiments of FIGS. 8-10 in that markings on at least the external surface 150 of the jaw 142 do not include numerical markings to represent distances from a reference point or axis. Instead, like the illustrated embodiments of FIGS. 5-7, the embodiments of FIG. 11-14 convey information through the use of geometric structures, figures, and/or designs.

In the embodiment of FIG. 11, the instrument 106 is illustrated in the left half of FIG. 11 with the external surface 150 in plan view, having markings 290, 292, 294 disposed on the external surface 150. According to certain embodiments, both jaws 140, 142 may have a marking, such as the markings 290, 292, 294, disposed on their respective external surfaces 148, 150.

The markings 290, 292, 294 are arranged on or formed on the external surface 150 such that at least the marking 294 is aligned with the at least one light sensor 122. In particular, where the at least one light sensor 122 is an array of light sensors 122, the marking 294 disposed on the external surface 150 corresponds to the center of the array. In fact, where the at least one light sensor 122 is a linear array of light sensors 122, the marking 294 is disposed on the external surface 150 at the middle of the linear array. The marking 290 is disposed on the external surface 150 at a first end of the array of light sensors 122, while the marking 292 is disposed on the external surface 150 at a second end of the array of light sensors 122. The markings 290, 292 are thus disposed to either side of the marking 294 because the marking 294 corresponds to the center or middle of the array of light sensors 122.

All three of the markings 290, 292, 294 may be of a particular geometric structure, design, or shape. As illustrated, the markings 290, 292, 294 may be rectangular boxes, which rectangular boxes may further be approximately square as illustrated. It is not necessary that all three markings use the same geometric structure, design, or shape. For example, the center marking 294 may be a different marking (e.g., circle) than the markings 290, 292 to either side. Moreover, the different structures, designs, or shapes may be carried over to the graphical interface to facilitate the correlation of the markings 290, 292, 294 on the jaw 142 with the graphical interface. While the markings 290, 292, 294 have been spaced from each other so that each geometric structure, design, or shape appears separately, the markings 290, 292, 294 may instead have been disposed on the surface 150 such that the outer markings 290, 292 abut the center marking 294 on either side of the center marking 294.

As is also illustrated in FIG. 11, each of the markings 290, 292, 294 may include an alphanumeric indicator associated with the marking 290, 292, 294. For example, the marking 292 may be associated with “A”, the marking 294 with “B”, and the marking 292 with “C”. This information may be carried over to the graphical interface to facilitate the correlation of the markings 290, 292, 294 on the jaw 142 with the graphical interface.

The visual display 160 is illustrated in the right half of FIG. 11, and may include a live image 300 of the surgical field 102 and a graphical interface 302. The controller 124 may combine the live image 300, as received from a camera or scope, with the graphical interface 302 provide an integrated image that includes both a visual image of the surgical field with information derived from the light sensor(s) 122. In particular, the controller 124 may determine a position of a tissue relative to the at least one light sensor 122 based on a signal from the at least one light sensor 122, and then control the visual display to display the graphical interface 302 that provides an image 304 corresponding to the position of the tissue between the jaws 140, 142.

More particularly, the controller 124 may control the visual display 160 to display a graphical interface 302 including at least one marking 306, 308, 310 corresponding to the at least one marking 290, 292, 294 on the external surface 150 of the second jaw 142 in combination with the image 300 corresponding to the tissue disposed between the first and second jaws 140, 142. As noted above, each marking 306, 308, 310 may be associated with one of the markings 290, 292, 294, and may be of a common structure, design, or shape with the markings 290, 292, 294. In the embodiment illustrated, it may appear that the regions or zones 306, 308, 310 are distinct from each other by one or more markings that brake up the image 304 into three regions, even though the regions or zones 306, 308, 310 are not spaced as the markings 290, 292, 294 so as to appear separately.

In the embodiment illustrated in FIG. 11, the image 304 also includes at least two different regions 312, 314. The region 312 includes the area between the dashed lines, and this region may represent a first tissue type, e.g., a blood vessel. The region 314 includes the areas outside the dashed lines, and this region may represent a second tissue type, e.g., adipose tissue. The two regions may be differentiated in the image 304 through the use of different colors; for example, the region 312 may be filled in red, while the region 314 may be filled in white. Other schemes may be used to differentiate the different regions apart (e.g., different shades of a single color), and the regions may include a single region (e.g., corresponding to only adipose is disposed between the jaws 140, 142 of the instrument 106) or more than two regions (e.g., a ureter, a blood vessel, and adipose tissue).

Other information may be combined with the graphical interface 302. For example, the width of the vessel may be displayed at one end or the other of the graphical interface 302. However, according to certain embodiments, it may be sufficient for the user to be able to correlate the location of the region 312 within the interface 302 with the marking 290, 292, 294 so as to be able to identify whether or not a particular tissue is near or at the center of the jaws 140, 142.

In the embodiment of FIG. 12, a single marking 324 may be arranged on or formed on the external surface 150 such that the marking 324 is aligned with the at least one light sensor 122. In particular, where the at least one light sensor 122 is an array of light sensors 122, the marking 324 may be disposed on the external surface 150 corresponding to the center of the array. In fact, where the at least one light sensor 122 is a linear array of light sensors 122, the marking 324 is disposed on the external surface 150 at the middle of the linear array. The marking 324 may be of a particular geometric structure, design, or shape, so as to permit quick localization of the center of the array, and thus quick correlation of the marking 324 on the jaw 142 with the representation on the graphical interface.

Markings 326, 328 may also be provided on the surface 150, one disposed to one side of the marking 324 and another disposed to the opposite side of the marking 324. The length of these longitudinally-oriented markings 326, 328 may indicate the length of the sensor array, similar to the markings of the embodiment of FIG. 10. This may also facilitate in the correlation of the markings 324, 326, 328 and the graphical interface.

The visual display 160 is illustrated in the right half of FIG. 12, and may include a live image 330 of the surgical field 102 and a graphical interface 332. The controller 124 may combine the live image 330, as received from a camera or scope, with the graphical interface 332 to provide an integrated image that includes both a visual image of the surgical field with information derived from the light sensor(s) 122. In particular, the controller 124 may determine a position of a tissue relative to the at least one light sensor 122 based on a signal from the at least one light sensor 122, and then control the visual display to display the graphical interface 332 that provides an image 334 corresponding to the position of the tissue between the jaws 140, 142. A scale 336 with graduated distance demarcations may also be provided to permit a visual estimation of the width of different regions of the image 334.

In the embodiment illustrated in FIG. 12, similar to other embodiments discussed above, the image 334 includes at least two different regions 338, 340. The region 338 includes the area between the dashed lines, and this region may represent a first tissue type, e.g., a ureter. The region 340 includes the areas outside the dashed lines, and this region may represent a second tissue type, e.g., adipose tissue. The two regions may be differentiated in the image 334 through the use of different colors, for example, the region 338 may be filled in red, while the region 340 may be filled in white. Other schemes may be used to differentiate the different regions apart (e.g., different shades of a single color), and the regions may include a single region (e.g., corresponding to only adipose is disposed between the jaws 140, 142 of the instrument 106) or more than two regions (e.g., a ureter, a blood vessel, and adipose tissue).

Other information may be combined with the graphical interface 332. For example, the width of the vessel may be displayed at one end or the other of the graphical interface 332. However, according to certain embodiments, it may be sufficient for the user to be able to correlate the location of the region 338 within the interface 332 so as to be able to identify whether or not a particular tissue is near or within the center of the jaws 140, 142.

To further facilitate the identification and correlation of the information on the graphical interface 332 with the markings 324, 326, 328, the graphical interface 332 may include a tissue indicator 342, The indicator 342 may include one or more markings that move along the image 334 as the region 338, for example, moves along the image 334 between the ends of the image 334. As illustrated, the indicator 342 includes two triangular markings, like arrow heads, that move along the image 334, one above the image 334 and one below the image 334. This may provide a graphical way for the user to identify the location of a particular region of tissue 338 on the image 334, and the correlate that information to the jaws 140, 142 via the markings 324, 326, 328. The tissue indicator 342 may even identify a particular subregion (e.g., an approximate longitudinal center) within the region of tissue 338 on the image 334.

As illustrated, the tissue indicator 342 may be configurable to convey not only the location of a region (or subregion) of tissue 338 on the image 334, but also a characteristic of the tissue. For example, the tissue indicator 342 may include an alphanumeric character or character that is associated with a tissue type. As illustrated, the tissue indicator 342 includes a “U” disposed transversely between the two triangular arrow heads, and the “U” may be associated with “ureter”. As such, the indicator 342 conveys a location of a ureter, but it also provides an indicator that differentiates the ureter from other tissue (e.g., a blood vessel). In a similar way, a blood vessel may be indicated as “BV” (for “blood vessel”) or “V” (for “vascular”), and other tissues may be represented as letters, numbers, or combinations thereof.

It will be recognized that while an alphanumeric character or character has been used in the illustration of FIG. 12, a geometric structure, design, or shape may be used instead. For example, a triangle may be substituted for the “U” to represent a ureter, a square may be used to represent a blood vessel, and so on. While a preferred embodiment that uses geometric structure, design, or shape in the tissue indicator 342 would use a geometric structure, design, or shape other than the marking 324 to avoid confusion as to the information being conveyed by the indicator 342 (i.e., that the geometric structure/tissue indicator 342 does not, in fact, show only the center of the array associated with marking 324), according to other embodiments, a circle may be used as the marking 324 and in the indicator 342.

FIG. 13 illustrates a still further embodiment that has similarities to the embodiment of FIG. 12. In particular, the embodiment of FIG. 13 uses a marking 354 in the form of a geometric structure, design, or shape (e.g., a circle) disposed on the surface 150 of the jaw 142 to indicate the center of a light sensor array. Unlike the embodiment of FIG. 12, the embodiment of FIG. 13 does not use longitudinal markings to either side of the marking 354. Instead, the embodiment of FIG. 13 uses a plurality of transverse markings 356 to indicate the length of the light sensor array, in that the markings 356 are disposed only on the region on the surface 150 approximately coextensive with the light sensor array. The markings 356 may provide an additional assistance to correlation of the tissue image that is part of the graphical interface with the jaws 140, 142, as explained below.

The visual display 160 is illustrated in the right half of FIG. 13, and may include a live image 330 of the surgical field 102 and a graphical interface 358. The controller 124 may combine the live image 330, as received from a camera or scope, with the graphical interface 358 to provide an integrated image that includes a visual image of the surgical field with information derived from the light sensor(s) 122. In particular, the controller 124 may determine a position of a tissue relative to the at least one light sensor 122 based on a signal from the at least one light sensor 122, and then control the visual display to display the graphical interface 358 that provides an image 360 corresponding to the position of the tissue between the jaws 140, 142.

In the embodiment illustrated in FIG. 13, similar to other embodiments discussed above, the image 360 includes at least two different regions 362, 364. The region 362 includes the area between the dashed lines, and this region may represent a first tissue type, e.g., a ureter or a blood vessel. The region 364 includes the areas outside the dashed lines, and this region may represent a second tissue type, e.g., adipose tissue. The two regions 362, 364 may be differentiated in the image 360 through the use of different colors; for example, the region 362 may be filled in red, while the region 364 may be filled in white.

The interface 358 may also include a geometric marking 366 and other markings 368. These markings 366, 368 correspond to the markings 354, 356 disposed on the surface 150 of the jaw 142 of the instrument or tool 106. The correspondence of the markings 366, 368 with the markings 354, 356 may assist in transferring or conveying information regarding the tissue disposed between the jaws 140, 142 to the user. In particular, the same geometric structure, design, or shape (i.e., a circle) has been used for the marking 354 and the marking 366 to better transfer or convey information to the user. In addition, the markings 356, 368 may be used to provide information about the relative location and size of the tissues displayed in the tissue image 260 and between the jaws 140, 142. In embodiments where the distance between the markings 356 is known, the distance between the markings 368 may be used to determine or approximate the size of tissue regions display in the image 360 (and thus the interface 358). The markings 366, 368 may be combined with other markings, such as may indicate the end or tip of the jaw 142, as well to transfer or convey additional information to the user. See FIG. 8, 9, or 14.

Otherwise, the discussion above relative to the embodiment of FIG. 12 (and the other embodiments above) may be generally applicable to the embodiment of FIG. 13 as well.

FIG. 14 illustrates still another embodiment that has similarities to the embodiments of FIGS. 12 and 13, for example. In particular, the embodiment of FIG. 14 uses a marking 374 in the form of a geometric structure, design, or shape (e.g., a circle) to indicate the center of a light sensor array. Unlike the embodiments of FIGS. 12 and 13, the embodiment of FIG. 14 does not use longitudinal or transverse markings to indicate the length of the light sensor array. Instead, a marking 376 in the form of a further geometric structure (a rectangle, as illustrated) is disposed on the surface 150 approximately coextensive with the light sensor array. The markings 366 may provide an additional assistance to correlation of the tissue image that is part of the graphical interface with the jaws 140, 142.

The visual display 160 is illustrated in the right half of FIG. 14, and may include a live image 330 of the surgical field 102 and a graphical interface 378. The controller 124 may combine the live image, as received from a camera or scope, with the graphical interface to provide an integrated image that includes both a visual image of the surgical field with information derived from the light sensor(s) 122. In particular, the controller 124 may determine a position of a tissue relative to the at least one light sensor 122 based on a signal from the at least one light sensor 122, and then control the visual display to display the graphical interface that provides an image 380 corresponding to the position of the tissue between the jaws 140, 142.

In the embodiment illustrated in FIG. 14, similar to other embodiments discussed above, the image 380 includes at least two different regions 382, 384. The region 382 includes the area between the dashed lines, and this region may represent a first tissue type, e.g., a ureter or a blood vessel. The region 384 includes the areas outside the dashed lines, and this region may represent a second tissue type, e.g., adipose tissue. The two regions 382, 384 may be differentiated in the image 380 through the use of different colors, for example, the region 382 may be filled in red, while the region 384 may be filled in white.

The interface 378 may also include a geometric marking 386 and other markings 388, 390. These markings 386, 388 correspond to the markings 374, 376 disposed on the surface 150 of the jaw 142 of the instrument or tool 106. The correspondence of the markings 386, 388 with the markings 374, 376 may assist in transferring or conveying information regarding the tissue disposed between the jaws 140, 142 to the user. In particular, the same geometric structure, design, or shape (i.e., a circle) has been used for the marking 374 and the marking 386 to better transfer or convey information to the user. In addition, the markings 376, 388 may be used to provide information about the relative location and size of the tissues displayed in the tissue image 280 and between the jaws 140, 142. The graphical interface may optionally include an orientation marking 390, similar to that shown in FIGS. 8 and 9 in the form of a rounded end corresponding to the tip of the jaw(s) 140, 142, so that the user may better correlate the marking(s) 374, 376 with the graphical interface 378.

Otherwise, the discussion above relative to the embodiment of FIGS. 12 and 13 (and the other embodiments above) may be generally applicable to the embodiment of FIG. 14 as well.

Although FIGS. 5-14 illustrate mainly embodiments wherein the markings on an instrument or tool are used with a system that references the positions of the tissues between the jaws of the instrument or tool, other embodiments may instead include markings on the instrument or tool to be used with a system that references the positions of tissue(s) proximate to the jaws of an instrument or tool where the tissue(s) is/are not between the jaws of such an instrument or tool. Such an embodiment may be particularly useful with reflectance-based systems, illustrated in FIGS. 3 and 4, where the emitter and sensor are positioned to detect tissues that are proximate to the end or tip of the jaws, as opposed to between the jaws. Such an embodiment may still include a marking on the jaws, but the graphical interface may include a tissue image that includes a jaw indicator to correlate the position of the end or tip of the jaw with the information regarding the tissue proximate (or distant) to the end or tip of the jaw. Further embodiments may include markings that permit distances of tissues proximate to the end or tip of the jaws to be displayed, in addition to markings that permit the information displayed regarding tissues between the jaws to be correlated with markings on an external surface of the jaws. Such embodiments are also within the scope of the present disclosure.

Having discussed various structures of the surgical system and its modes of operation, additional details regarding the sensor, the controller and other ancillary equipment are now provided.

While the foregoing graphical interface may be used with the light emitter 120 and light sensor 122 that together define the sensor, it will be recognized that the graphical interface may be used with other sensors as well As mentioned above, the graphical interface may be used with an ultrasonic sensor, for example. The most preferred system includes the graphical interface, the light emitter 120 and the light sensor 122, however. Consequently, further comments regarding the light emitter 120 and light sensor 122 are included below.

The light emitter 120 may include one or more elements, as referenced above. According to an embodiment schematically illustrated in FIG. 2, the light sensor 122 may include a first light emitter 120-1, a second light emitter 120-2, and a third light emitter 120-3. All of the light emitters may be adapted to emit light at a particular wavelength (e.g., 660 nm), or certain emitters may emit light at different wavelengths than other emitters. Each light emitter may be a light emitting diode, for example.

As to those embodiments wherein the light emitter 120 is in the form of an array including one or more light emitting diodes, as is illustrated in FIG. 2, the diodes may be arranged in the form of a one-dimensional, two-dimensional, or three-dimensional array. An example of a two-dimensional array may include disposing the diodes in a plurality of rows and columns in a single plane. A further example of a two-dimensional array may include disposing the diodes along a line on or in a curved surface. A three-dimensional array may include diodes disposed in more than one plane, such as in a plurality of rows and columns on or in a curved surface.

The light sensor 122 also may include one or more elements. Again, according to the embodiment illustrated in FIG. 2, the light sensor 122 may include a first light sensor 122-1, a second light sensor 122-2, an n-th light sensor 122-n, and so on. As was the case with the light emitters 120-1, 120-2, 120-3, the light sensors 122-1, 122-2, 122-3 may be arranged in an array, and the discussion about the arrays above applied with equal force here.

In fact, where the array of light sensors 122 includes a row of light sensors (such as in FIG. 2), the array 122 may be referred to in the alternative as a linear array. The individual light sensors of the array 122 may be disposed adjacent each other, or the light sensors may be spaced from each other. It may even be possible for the individual light sensors that define a row of light sensors to be separated from each other by light sensors that define a different row or column of the array. According to a particular embodiment, however, the array may comprise a charge coupled device (CCD), and in particular linear CCD imaging device comprising a plurality of pixels. As a further alternative, a CMOS sensor array may be used.

While the arrangement of the light emitter 120 and the light sensor 122 may vary between the transmittance-based embodiment of FIG. 2 and the reflectance-based embodiments of FIGS. 3 and 4, it is equally true that the light emitter 120 and the light sensor 122 of the reflectance-based embodiments may involve a plurality of elements.

Contrasting the arrangement illustrated in FIGS. 3 and 4 with that of FIG. 2, the light emitter 120 and light sensor 122 are disposed generally facing in a common direction (i.e., the direction of the tissue sample of interest). This does not require the emitter 120 and the sensor 122 to be generally disposed in a common plane, although this is preferred. According to certain embodiments, the emitter 120 and sensor 122 may be formed integrally (i.e., as one piece) with a surgical instrument 106 (see FIGS. 3 and 4), although other options are possible, as discussed below. In this manner, light emitted by the emitter 120 and scattered by the tissue of interest may be captured by the light sensor 122.

Further, it is believed that the spacing between the emitter 120 and the sensor 122 may influence the light received by the sensor 122. As presently understood, after photons leave the emitter 120 in contact with tissue, an ensemble of independent photons return to the surface and reach the sensor 122. Some of the detected photons travel a short distance from the plane of the emitter and detector and exit at the site of the sensor 122, while some photons travel farther into the tissue before exiting at the surface without being absorbed (photons that are absorbed cannot contribute to the photocurrent). Path length distributions and the penetration depth of photons that reach the sensor 122 vary as a function of emitter-sensor separation, with maximum effective photon depth penetration values several times greater than the physical emitter-sensor separation. For example, it has been determined that a spacing between the emitter 120 and the sensor 122 of 5 mm may permit detection of vessels from 0 mm to 12 mm from the surface of the tissue.

Changes in blood volume, due to differences in systolic and diastolic pressures within a tissue-embedded artery, affect the relative number of long-traveling photons that survive and reach the sensor 122. The temporally observed difference in the number of long-traveling photons that results from the presence of an artery in the photon trajectory is responsible for the pulsatile (AC) signal. For a small source-detector separation, detected photons traversing the shorter distances are less exposed to the cycling blood of an artery at a greater depth below the tissue surface, and therefore survive with a more uniform likelihood between systolic and diastolic conditions. With an increased source-detector separation, a higher percentage of photons that reach the sensor 122 will be long-traveling photons, resulting in larger detected pulse amplitudes. Therefore, it is believed that increasing the spacing between the emitter 120 and the sensor 122 may permit the light to penetrate even deeper into the tissue, permitting vessel detection at even greater depths.

It is further believed that adjusting the angle of the emitter 120 and/or sensor 122 may provide a similar effect. That is, similar to the way in which a change in the linear distance between the emitter 120 and the sensor 122 allows for the sampling of a different proportion of long-traveling photons at the surface sensor 122, a variation in angle of the emitter 120 and/or sensor 122 can change the depth and the distance to which the photons travel before being sampled by the sensor 122. Consequently, changes in the angle of the emitter and/or sensor are believed to permit the depth at which vessels can be detected by the instrument 106 to be varied.

Thus, according to the embodiments described herein, the emitter 120 and sensor 122 may be disposed to be mounted in a fixed relationship to each other, or a moveable or adjustable relationship. In particular, FIG. 3 illustrates an embodiment wherein emitter 120 and sensor 122 are at a fixed spacing relative to each other, and also have a fixed angular relationship between the emitter 120 and the sensor 122. Such an embodiment would permit the user to be confident that the vessels detected are within, for example, 12 mm from the working end 104 of the instrument 106. By contrast, the embodiment of FIG. 4 has the emitter 120 mounted in a first jaw 140 of the instrument 106 and the sensor 122 mounted in a second jaw 142 of the instrument 106. Such an embodiment would permit the user to vary the depth of detection simply by varying the distance between the jaws 140, 142 of the instrument 106: with the jaws 140, 142 closed, the user may probe for shallow vessels (i.e., vessels disposed within 12 mm of the tissue surface), while with the jaws 140, 142 open, the user may probe for deeper vessels (i.e., vessels disposed greater than 12 mm below the tissue surface). According to the embodiment illustrated in FIG. 4, the control structure for operating the jaws 140, 142 may include a mechanism for modifying the distance between the jaws 140, 142 in a controlled fashion (e.g., in discrete increments) so that the user can determine the jaw spacing (and thus the detection depth) without visualization of the jaws 140, 142.

As mentioned above, the light emitter 120 of FIGS. 3 and 4 may include one or more elements. According to such an embodiment, all of the elements may be adapted to emit light at a particular wavelength (e.g., 660 nm), or certain elements may emit light at different wavelengths than other elements. It is believed that a system with multiple light emitters 120 and/or multiple sensors 122 will increase the signal-to-noise ratio and the spatial resolution compared to a system containing a single emitter 120 and sensor 122.

As to those embodiments wherein the light emitter 120 is in the form of an array including one or more light emitting diodes, the diodes may be arranged in the form of a one-dimensional, two-dimensional, or three-dimensional array. An example of a two-dimensional array may include disposing the diodes in a plurality of rows and columns in a single plane. Further example of a two-dimensional array may include disposing the diodes along a line on or in a curved surface. A three-dimensional array may include diodes disposed in more than one plane, such as in a plurality of rows and columns on or in a curved surface.

In addition, the light sensor 122 may include a mechanism for physically excluding photons reaching the sensor 122 from a range of angles. This mechanism can consist of a mask or grated layer to physically filter any photons that are not reaching the sensor 122 at a nearly perpendicular angle. It has been observed that the mean depth penetration of the photons leaving the emitter 120 is equal to just over half the distance of source-detector separation (˜2.5 mm penetration for our 5 mm spacing). This mechanism will increase the proportion of long-traveling and deep penetrating photons that are received by the sensor 122 thus increasing the depth at which the vessels can be detected by the instrument.

As to all of the foregoing embodiments, the system 100 may include hardware and software in addition to the emitter 120, sensor 122, and controller 124. For example, where more than one emitter 120 is used, a drive controller may be provided to control the switching of the individual emitter elements. In a similar fashion, a multiplexer may be provided where more than one sensor 122 is included, which multiplexer may be coupled to the sensors 122 and to an amplifier. Further, the controller 124 may include filters and analog-to-digital conversion as may be required.

According to certain embodiments, the splitter 126 and the analyzer 128 may be defined by one or more electrical circuit components. According to other embodiments, one or more processors (or simply, the processor) may be programmed to perform the actions of the splitter 126 and the analyzer 128. According to still further embodiments, the splitter 126 and the analyzer 128 may be defined in part by electrical circuit components and in part by a processor programmed to perform the actions of the splitter 126 and the analyzer 128.

For example, the splitter 126 may include or be defined by the processor programmed to separate the pulsatile component from the non-pulsatile component. Further, the analyzer 128 may include or be defined by the processor programmed to determine the presence of (or to quantify the size of, for example) the vessel V within the region 102 proximate to the working end 104 of the surgical instrument 106 based on the pulsatile and/or the non-pulsatile component. The instructions by which the processor is programmed may be stored on a memory associated with the processor, which memory may include one or more tangible non-transitory computer readable memories, having computer executable instructions stored thereon, which when executed by the processor, may cause the one or more processors to carry out one or more actions.

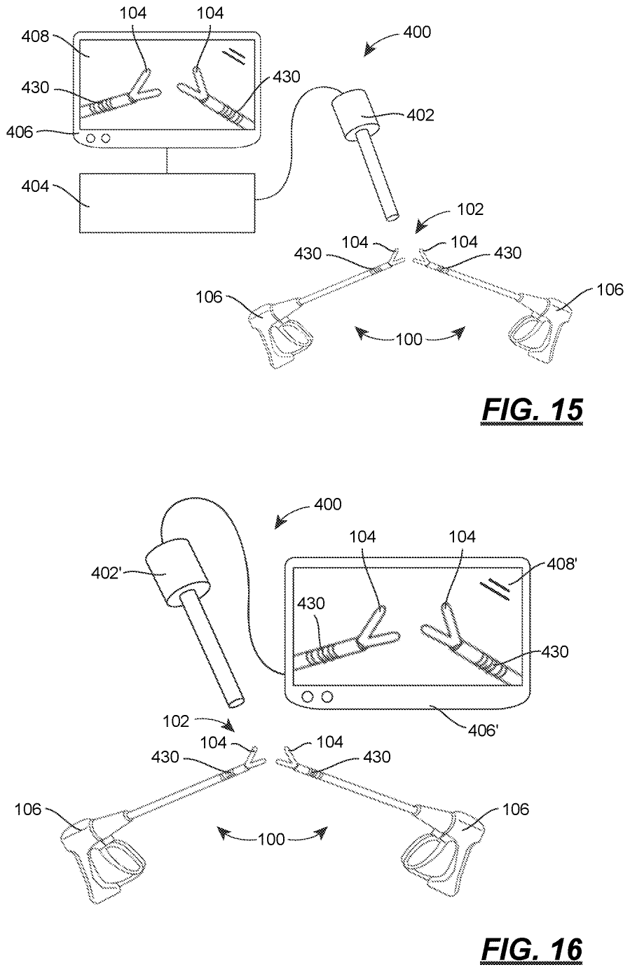

In addition to the foregoing, FIGS. 15 and 16 illustrate an embodiment of the surgical system 100 in combination with embodiments of a video system 400, such as may be used conventionally during minimally invasive surgery or laparoscopic surgery, for example. The video system 400 includes a video camera or other image capture device 402, a video or other associated processor 404, and a display 406 having a viewing screen 408.

As illustrated, the video camera 402 is directed at the region 102 proximate the working ends 104 of two surgical instruments 106. As illustrated, both of the surgical instruments 106 are part of an embodiment of a surgical system 100, such as illustrated in FIG. 1 and discussed above. The other elements of the surgical system 100 are omitted for ease of illustration, although it will be noted that elements of the system 100, such as the splitter 126 and the analyzer 128, may be housed in the same physical housing as the video processor 404.

The signal from the video camera 402 is passed to the display 406 via the video processor 404, so that the surgeon or other member of the surgical team may view the region 102 as well as the working ends 104 of the surgical instruments 106, which are typically inside the patient. Because of the proximity of the markings on the surface 150 of the jaws 140, 142, and thus the region 102, the markings are also visible on the display screen 408. As mentioned previously, this advantageously permits the surgeon to receive visual cues via the markings via the same display 406 and on the same display screen 408 as the region 102 and the working ends 104. This, in turn, limits the need of the surgeon to look elsewhere for the information conveyed via the markings.

FIG. 16 illustrates another embodiment of a video system 400 that can be used in conjunction with an embodiment of the surgical system 100. According to this embodiment, the video processor 404 is not disposed in a housing separate from the video camera 402′, but is disposed in the same housing as the video camera 402′. According to a further embodiment, the video processor 404 may be disposed instead in the same housing as the remainder of the display 406′ as the display screen 408′. Otherwise, the discussion above relative to the embodiment of the video system 400 illustrated in FIG. 15 applies equally to the embodiment of the video system 400 illustrated in FIG. 16.

While the combination of markings on a surface of the surgical instrument and the graphical interface, above, advantageously permits the surgeon or surgical team to view an output from the controller 124, it is possible to include other output devices, as illustrated in FIGS. 1, 15, and 16. For example, an alert may be displayed on a video monitor being used for the surgery (e.g., the display 406, 406′ in FIGS. 15 and 16), or may cause an image on the monitor to change color or to flash, change size or otherwise change appearance. In addition, one or more light emitting elements 430 may be disposed at the working end 104 of the surgical instrument 106 (see FIGS. 15 and 16), or at the proximal end 110 of the shaft 108 (including disposed on or attached to the grip or handle 112) to provide a visual indication or alarm. The auxiliary output may also be in the form of or include a speaker 502 that provides an auditory alarm. The auxiliary output also may be in the form of or may incorporate a safety lockout associated with the surgical instrument 106 that interrupts use of the instrument 106. For example, the lockout could prevent ligation or cauterization where the surgical instrument 106 is a thermal ligature device. As a still further example, the auxiliary output also may be in the form of a haptic feedback system, such as a vibrator 504, which may be attached to or formed integral with a handle or handpiece of the surgical instrument 106 to provide a tactile indication or alarm. Various combinations of these particular forms of the auxiliary output may also be used.

Where the surgical system 100 also includes one or more light emitting elements 430 disposed at the working end 103 or the proximal end 110 of the surgical instrument, the one or more light emitting elements 430 may be as disclosed in U.S. Pub. No. 2017/0367772, which is incorporated by reference in its entirety herein.

Further, the one or more light emitting elements 430 (as well as the light emitter 120 and the light sensor 122) may be attached (in the alternative, removably/reversibly (e.g., clip on) or permanently/irreversibly (e.g., attached with adhesive)) to the instrument or tool 106. The light emitting elements 430 may instead be formed integrally (i.e., as one piece) with the surgical instrument 106. As also stated, it is possible that the light emitting elements 430 may be attached to a separate instrument or tool that is used in conjunction with a surgical instrument or tool 106.

As noted above, the surgical instrument 106 may be a thermal ligature device in one embodiment illustrated in FIG. 1. In another embodiment, the surgical instrument 106 may simply be a grasper or grasping forceps having opposing jaws. According to still further embodiments, the surgical instrument may be other surgical instruments such as forceps, hemostats, sealer/divider, irrigators, surgical staplers, clip appliers, and robotic surgical systems, for example. According to still other embodiments, the surgical instrument may have no other function that to carry the graphical interface and sensor and to place them within a surgical field. The illustration of a single embodiment is not intended to preclude the use of the system 100 with other surgical instruments or tools 106.

In conclusion, although the preceding text sets forth a detailed description of different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, which is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. § 112(f).

1. A medical system, comprising:
a first jaw having an internal surface and a second, opposing jaw having an internal surface;
at least one light emitter disposed on the internal surface of one of the first and second jaws, and at least one light sensor disposed on the internal surface of one of the first and second jaws,
at least one of the first and second jaws having an external surface opposite the internal surface, the external surface having at least one marking disposed on the external surface, and the at least one marking aligned with the at least one light sensor;
at least one visual display; and
a controller coupled to the at least one light sensor and the at least one visual display, the controller configured to:
determine a position of a tissue relative to the at least one light sensor based on a signal from the at least one light sensor, and
control the at least one visual display to display a graphical interface comprising at least one marking corresponding to the at least one marking on the external surface of the at least one of the first and second jaws in combination with an image corresponding to the tissue disposed between the first and second jaws.

2. The medical system according to claim 1, wherein the at least one light sensor comprises an array of light sensors, and the at least one marking disposed on the external surface corresponds to a center of the array.

3. The medical system according to claim 2, wherein the array is a linear array, and the at least one marking disposed on the external surface corresponds to the middle of the linear array.

4. The medical system according to claim 2, wherein at least one marking disposed on the external surface comprises a first marking corresponding to a first end of the array, a second marking corresponding to a second end of the array, and a third marking corresponding to a center of the array.

5. The medical system according to claim 4, wherein the at least one marking disposed on the external surface comprises additional markings disposed between the first and third markings and the second and third markings, the additional markings being of different dimension than the first, second and third markings.

6. The medical system according to claim 4, wherein the first, second, and third markings are lines.

7. The medical system according to claim 4, wherein the third marking has a design superimposed on the third marking.

8. The medical system according to claim 4, where the at least one marking comprises a longitudinal line disposed to one side of the first, second, and third markings.

9. The medical system according to claim 4, wherein the at least one marking comprises a longitudinal line disposed along a midpoint of each of the first, second and third markings.

10. The medical system according to claim 4, wherein the at least one marking comprises a pair of longitudinal lines, one disposed to one side of the first, second, and third markings and one disposed to an opposite side of the first, second, and third markings.

11. The medical system according to claim 4, wherein the first marking is disposed closer to an end of one of the first and second jaws, and the second marking is disposed closer to a pivot between the first and second jaws, and the first marking is different from the second marking.

12. The medical system according to claim 4, wherein the first, second, and third markings are geometric figures.

13. The medical system according to claim 1, wherein the at least one visual display comprises a video monitor, a heads-up video display, a pair of smart glasses, and a video headset.

14. The medical system according to claim 1, wherein:
the first and second jaws are movably connected, the jaws movable between a first position with the internal surface of the first jaw proximate to the internal surface of the second jaw and a second position with the internal surface of the first jaw spaced from the internal surface of the second jaw.

15. The medical system according to claim 14, wherein the first and second jaws are pivotally connected.

16. The medical system according to claim 15, wherein the tissue is a vessel, such as a blood vessel.
